(19) United States
(12) Patent Application Publication
(10) Pub. No.: US 2008/0147028 A1
(43) Pub. Date: Jun. 19, 2008

(54) Deodorizing release liner for absorbent articles

(76) Inventors: Marie Luna, Appleton, WI (US); J. Gavin MacDonald, Decatur, GA (US); Jaeho Kim, Roswell, GA (US); Stephanie M. Martin, Woodstock, GA (US); Kelly D. Arehart, Roswell, GA (US); Bao Trong Do, Decatur, GA (US)

Correspondence Address: DORITY & MANNING, P.A., POST OFFICE BOX 1449, GREENVILLE, SC, 29602-1449, US

(21) Appl. No.: 11/640,463

(22) Filed: Dec. 15, 2006

(51) Int. Cl. A61F 13/15; A61L 9/00

(52) U.S. Cl. 604/359; 422/5

(57) ABSTRACT

A deodorizing release liner for use in absorbent articles, such as sanitary napkins, is provided. More specifically, one or more surfaces of the release liner are coated with an ink that contains an odor control agent capable of reducing odor associated with a bodily fluid (e.g., menses, urine, etc.). The release liner is initially positioned adjacent to an adhesive located on the absorbent article. To use the absorbent article, the liner may be peeled away from the adhesive and then discarded, either alone or in conjunction with a used absorbent article (e.g., in a container).

### BACKGROUND OF THE INVENTION

Absorbent feminine care articles, such as sanitary napkins, panty liners, labial pads, and other types of catamenial devices, are used to absorb menses and other body fluids. These absorbent products are used during a women's menstrual cycle or between menstrual cycles for light incontinence purposes. Regardless, the absorbent articles are primarily designed for a single use, after which they are discarded into a toilet pail or trash receptacle. Unfortunately, however, storage in a toilet pail located in a bathroom or in some other trash receptacle may rapidly result in the development of disagreeable odors. As such, a need currently exists for a method for reducing the odor produced by personal care absorbent articles, particularly after they are disposed.

### SUMMARY OF THE INVENTION

In accordance with one embodiment of the present invention, an absorbent article is disclosed that comprises a body portion that includes a liquid permeable topsheet, a generally liquid impermeable backsheet, and an absorbent core positioned between the backsheet and the topsheet. The absorbent article further comprises a release liner that defines a first surface and an opposing second surface, the first surface being disposed adjacent to an adhesive located on the absorbent article. The release liner is coated with an ink that contains an odor control agent.

In accordance with another embodiment of the present invention, a deodorizing release liner is disclosed that defines a first surface and an opposing second surface. An ink that contains an odor control agent is provided on the first surface of the liner and a coating that comprises a release agent is provided on the second surface of the liner.

In accordance with yet another embodiment of the present invention, a method for reducing the odor associated with a personal care absorbent article that contains a bodily fluid (e.g., urine, menses, etc.) is disclosed. The method comprises disposing the article and a release liner into a container. The release liner is coated with an ink that contains an odor control agent configured to adsorb a malodorous compound associated with the bodily fluid.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a top view of an absorbent article that may be formed in accordance with one embodiment of the present invention.

Repeat use of references characters in the present specification and drawing is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a deodorizing release liner for use in absorbent articles, such as sanitary napkins. More specifically, one or more surfaces of the release liner are coated with an ink that contains an odor control agent capable of reducing odor associated with a bodily fluid (e.g., menses, urine, etc.). The release liner is initially positioned adjacent to an adhesive located on the absorbent article. To use the absorbent article, the liner may be peeled away from the adhesive and then discarded, either alone or in conjunction with a used absorbent article (e.g., in a container).

### I. Release Liner

The releaser liner of the present invention may be constructed from any of a variety of materials as is known in the art. For example, the release liner may be formed from a paper (e.g. white Kraft paper), film, nonwoven web, etc. In one embodiment, for example, the release liner includes a film formed from a polymer, such as polyolefins (e.g., polyethylene, polypropylene, etc.), ethylene vinyl acetate, ethylene ethyl acrylate, ethylene acrylic acid, ethylene methyl acrylate, ethylene normal butyl acrylate, nylon, ethylene vinyl alcohol, polystyrene, polyurethane, and so forth. If desired, the release liner may be subjected to one or treatments that enhance the resulting durability of the deodorizing ink. For instance, because the deodorizing ink may be aqueous-based, the release liner may be subjected to a hydrophilic treatment to improve its affinity for the ink. For example, the release liner may be subjected to corona field that results in morphological and chemical modifications of the surface of the release liner. The term “corona field” generally refers to a corona field of ionized gas. The dose or energy density to which the substrate is exposed may range from about 1 to about 500 watt-minute per square foot (w-min/ft²), in some embodiments from about 15 to about 350 w-min/ft², and in some embodiments, from about 20 to about 80 w-min/ft². The corona field may be applied to the substrate under ambient temperature and pressure; however, higher or lower temperature and pressures may be used. Various suitable corona discharge treatments are described, for instance, in U.S. Pat. No. 4,283,291 to Lowther; U.S. Pat. No. 3,754,117 to Walter; U.S. Pat. No. 3,880,966 to Zimmerman, et al.; and U.S. Pat. No. 3,471,597 to Schirmer, which are incorporated herein in their entirety by reference thereto for all purposes. In addition to or in conjunction with corona discharge treatment, the release liner may also be applied with a hydrophilic compound. One class of suitable hydrophilic compounds includes polysaccharides, which are described in more detail below.

The release liner may have any desired shape or dimension. For instance, the liner may have a rectangular shape having a length of about 10 to about 20 centimeters and a width of about 1 to about 10 centimeters. The thickness of the release liner may generally vary depending upon the desired use. For example, in most embodiments of the present invention, the release liner has a thickness of about 50 micrometers or less, in some embodiments from about 1 to about 40 micrometers, in some embodiments from about 2 to about 35 micrometers, and in some embodiments, from about 5 to about 30 micrometers.

A. Deodorizing Ink

A deodorizing ink is applied to one or more surfaces of the release liner for reducing odor. The deodorizing ink contains an odor control agent that is capable of adsorbing malodorous compounds generated during use of an absorbent article. Any of a variety of odor control agents may generally be employed in the present invention. Activated carbon particles, for instance, may be a suitable odor control agent for use in the present invention. Activated carbon particles may be derived from a variety of sources, such as from sawdust, wood, charcoal, peat, lignite, bituminous coal, coconut shells, etc. The particles may be in the shape of a sphere, crystal, rod, disk, tube, string, etc. The average size (e.g., diameter or width) of the activated carbon particles is suitable about 50 micrometers or less, in some embodiments about 25 micrometers or less, and in some embodiments, from about 0.1 to about 10 micrometers. Without intending to be limited by theory, it is believed that particles having such a small size and high corresponding surface area may improve the adsorption capability for many malodorous compounds. Some suitable forms of activated carbon and techniques for formation thereof are described in U.S. Pat. No. 5,693,385 to Parks; U.S. Pat. No. 5,834,114 to Economy, et al.; U.S. Pat. No. 6,517,906 to Economy, et al.; U.S. Pat. No. 6,573,212 to McCrae, et al., as well as U.S. patent application Publication Nos. 2002/0141961 to Falat, et al. and 2004/0166248 to Hu, et al., all of which are incorporated herein in their entirety by reference thereto for all purposes.

The odor control agent may also include inorganic nanoparticles having an average particle size (e.g., diameter or width) of about 5 micrometers or less, in some embodiments about 1 micrometer or less, in some embodiments about 100 nanometers or less, in some embodiments from about 1 to about 50 nanometers, and in some embodiments, from about 2 to about 25 nanometers. If desired, the nanoparticles may also be relatively nonporous or solid. That is, the nanoparticles may have a pore volume that is less than about 0.5 milliliters per gram (ml/g), in some embodiments less than about 0.4 milliliters per gram, in some embodiments less than about 0.3 ml/g, and in some embodiments, from about 0.2 ml/g to about 0.3 ml/g. Without intending to be limited by theory, it is believed that the solid nature, i.e., low pore volume, of the nanoparticles may enhance the uniformity and stability of the nanoparticles, without sacrificing their odor adsorption characteristics.

Suitable inorganic oxide nanoparticles include, for instance, silica, alumina, zirconia, magnesium oxide, titanium dioxide, iron oxide, zinc oxide, copper oxide, zeolites, clays (e.g., smectite clay), combinations thereof, and so forth. Various examples of such nanoparticles are described in U.S. patent application Publication Nos. 2003/0203009 to MacDonald; 2005/0084412 to MacDonald, et al.; and 2005/0085144 to MacDonald, et al., which are incorporated herein in their entirety by reference thereto for all purposes. If desired, the nanoparticles may be selected to have a zeta potential that facilitates ionic bonding with certain compounds (e.g., odor control agent, malodorous compounds, etc.), a substrate, and so forth. For example, the nanoparticles may possess a negative zeta potential, such as less than about 0 millivolts (mV), in some embodiments less than about −10 mV, and in some embodiments, less than about −20 mV. Examples of nanoparticles having a negative zeta potential include silica nanoparticles, such as Snowtex-C, Snowtex-O, Snowtex-PS, and Snowtex-OXS, which are available from Nissan Chemical of Houston, Tex. Alternatively, the nanoparticles may have a positive zeta potential, such as greater than about 0 millivolts, in some embodiments greater than about +20 millivolts (mV), in some embodiments greater than about +30 mV, and in some embodiments, greater than about +40 mV. The nanoparticles may, for instance, be formed entirely from a positively charged material, such as alumina. Examples of commercially available alumina nanoparticles include, for instance, Aluminasol 100, Aluminasol 200, and Aluminasol 520, which are available from Nissan Chemical Industries Ltd. The positive zeta potential may also be imparted by a continuous or discontinuous coating present on the surface of a core material. In one particular embodiment, for example, the nanoparticles are formed from silica nanoparticles coated with alumina. A commercially available example of such alumina-coated silica nanoparticles is Snowtex-AK, which is available from Nissan Chemical of Houston, Tex.

Although the nanoparticles themselves possess a certain degree of odor reducing properties, they may nevertheless be modified with a transition metal to improve their odor control properties. Without being limited by theory, it is believed that the transition metal provides one or more active sites for capturing and/or neutralizing a malodorous compound. The active sites may be free, or may be weakly bound by water molecules or other ligands so that they are replaced by a malodorous molecule when contacted therewith. In addition, the nanoparticles still have the large surface area that is useful in adsorbing other malodorous compounds. Examples of some suitable transition metals that may be used in the present invention include, but are not limited to, scandium, titanium, vanadium, chromium, manganese, iron, cobalt, nickel, copper, zinc, and so forth. Single metallic, as well as dinuclear, trinuclear, and cluster systems may be used. The ratio of the transition metal to the nanoparticles may be selectively varied to achieve the desired results. In most embodiments, for example, the molar ratio of the transition metal to the nanoparticles is at least about 10:1, in some embodiments at least about 25:1, and in some embodiments, at least about 50:1.

Due to the addition of the transition metal, the modified nanoparticles may sometimes exhibit a Zeta Potential that is different than the Zeta Potential of the nanoparticles prior to modification. The addition of positively-charged metal ions may, for instance, increase the Zeta Potential of the unmodified nanoparticles by at least about 1.0 millivolt, and in some embodiments, by at least about 5.0 millivolts. Of course, the particular difference in Zeta Potential, if any, is related in part to the quantity and type of transition metal employed. For instance, the addition of a dilute solution of copper chloride to a silica nanoparticle solution may result in a change in Zeta Potential of the silica suspension from −25 millivolts to a higher Zeta Potential, such as in the range of about −5 millivolts to −15 millivolts.

The transition metal may be applied to the nanoparticles in a variety of ways. For instance, nanoparticles may simply be mixed with a solution containing the appropriate transition metal in the form of a salt, such as those containing a copper ion (Cu⁺²), iron (III) ion (Fe⁺³), and so forth. Such solutions are generally made by dissolving a metallic compound in a solvent resulting in free metal ions in the solution. Generally, the metal ions are drawn to and adsorbed onto the nanoparticles due to their electric potential differences, i.e., they form an “ionic” bond. In many instances, however, it is desired to further increase the strength of the bond formed between the metal and nanoparticles through the formation of a coordinate and/or covalent bond. Although ionic bonding may still occur, the presence of coordinate or covalent bonding may have a variety of benefits, such as reducing the likelihood that any of the metal will remain free during use (e.g., after washing). Further, a strong adherence of the metal to the nanoparticles also optimizes odor adsorption effectiveness.

Numerous techniques may be utilized to form a stronger bond between the transition metal and nanoparticles. For example, silica sols are generally considered stable at a pH of greater than about 7, and particularly between a pH of 9-10. When dissolved in water, salts of transition metals are acidic (e.g., copper chloride has a pH of approximately 4.8). Thus, when such an acidic transition metal salt is mixed with a basic silica sol, the pH is lowered and the metal salt precipitates on the surface of the silica particles. This compromises the stability of the silica particles. Further, at lower pH values, the number of silanol groups present on the surface of the silica particles is reduced. Because the transition metal binds to these silanol groups, the capacity of the particles for the transition metal is lowered at lower pH values. Thus, to ameliorate the pH-lowering affect caused by the addition of an acidic transition metal salt (e.g., copper chloride), certain embodiments of the present invention employ selective control over the pH of the silica particles during mixing with the transition metal.

The selective control over pH may be accomplished using any of a variety of well-known buffering systems known in the art. One such buffering system utilizes urea thermal decomposition (i.e., pyrolysis) to increase pH to the desired value. The pyrolysis of urea is well known, and has been described in, for instance, Study of the Urea Decomposition (Pyrolysis) Reaction and Importance to Cyanuric Acid Production, Peter M. Shaber, et al., American Laboratory (August 1999), which is incorporated herein in its entirety by reference thereto for all purposes. For instance, to initiate the pyrolysis reaction, urea is first heated to its melting point of approximately 135° C. With continued heating to approximately 150° C., the urea is vaporized (Eq. 1) and is then decomposed into ammonia and isocyanic acid (Eq. 2). The urea also reacts with the isocyanic acid byproduct to form biuret (Eq. 3).

H₂N—CO—NH₂(m)+heat [symbol] H₂N—CO—NH₂(g) (1)

H₂N—CO—NH₂(g)+heat [symbol] NH₃(g)+HNCO(g) (2)

H₂N—CO—NH₂(m)+HNCO(g) [symbol] H₂N—CO—NH—CO—NH₂(s) (3)

### Upon further heating, e.g., to about 175° C., the biuret referenced above reacts with isocyanic acid to form cyanuric acid and ammonia (Eq. 4), as well as ammelide and water (Eq. 5).

H₂N—CO—NH—CO—NH₂(m)+HNCO(g) [symbol] CYA(s)+NH₃(g) (4)

H₂N—CO—NH—CO—NH₂(m)+HNCO(g) [symbol] ammelide(s)+H₂O(g) (5)

As the temperature is further increased, other reactions begin to occur. For instance, biuret may decompose back into urea and isocyanic acid. The urea produced is unstable at higher temperatures, and thus, will further decompose into ammonia and isocyanic acid. Urea and the byproducts of the pyrolysis reaction will continue to react and further decompose as the reaction mixture is heated.

One advantage of using urea decomposition to control the pH of the transition metal/silica mixture is the ability to easily manipulate pH as the metal and silica are mixed together. For instance, as indicated above, the pyrolysis of urea produces ammonia (NH₃) as a byproduct. In some embodiments of the present invention, the presence of this ammonia byproduct may be used to increase the pH of the transition metal/silica mixture to the desired level. The amount of ammonia present in the mixture may be easily controlled by selectively varying the amount of urea reactant and the temperature to which the urea is heated. For instance, higher pyrolysis temperatures generally result in a greater amount of resulting ammonia due to the greater extent to which the urea and its byproducts are decomposed.

Besides urea decomposition, other well-known buffering systems may also be employed in the present invention to increase the pH of the transition metal/silica mixture to the desired level. For instance, in one embodiment, the buffering system may use an alkali metal bicarbonate and an alkali metal carbonate in a certain molar ratio. The alkali metal cations may be, for instance, sodium and/or potassium. In one particular embodiment, the buffering system employs sodium carbonate (Na₂CO₃) and sodium bicarbonate (NaHCO₃). In other embodiments of the present invention, the buffering system may simply involve adding a certain amount of a basic compound to the mixture, such as sodium hydroxide, potassium hydroxide, ammonium hydroxide, and so forth. Regardless of the technique for increasing the pH of the transition metal/silica mixture, it is believed that the adjustment allows stronger bonds to be formed between the transition metal and silica particles. Specifically, without intending to be limited by theory, it is believed that the transition metal is capable of forming covalent bonds with the silanol groups present on the silica particle surface. In addition, the higher pH increases the number of silanol groups available for binding and reduces salt precipitation, thereby enhancing bonding efficiency. Of course, due to the opposite charge of the transition metal and some types of silica particles, some binding via electrostatic attraction will also be present.

Apart from pH adjustment, other techniques may also be utilized to further enhance the strength of the bonds formed between the transition metal and the nanoparticles, such as the use of coupling agents (e.g., organofunctional silanes), bifunctional chelating agents (e.g., EDTA), and so forth. Examples of such techniques are described in more detail in U.S. patent application Publication Nos. 2005/0084438 to Do, et al.; 2005/0084474 to Wu, et al.; and 2005/0084464 to McGrath, et al., which are incorporated herein in their entirety by reference thereto for all purposes.

If desired, more than one type of transition metal may be bound to a particle. This has an advantage in that certain metals may be better at removing specific malodorous compounds than other metals. Likewise, different types of nanoparticles may be used in combination for effective removal of various malodorous compounds. In one embodiment, for instance, copper-modified silica nanoparticles are used in combination with manganese-modified silica nanoparticles. By using two different nanoparticles in combination, numerous malodorous compounds may be more effectively removed. For example, the copper-modified particle may be more effective in removing sulfur and amine odors, while the manganese-modified particle may be more effective in removing carboxylic acids.

The odor control agent may also employ an odor-reducing anthraquinone having the following general formula:

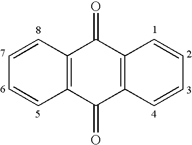

The numbers 1-8 shown in the general formula represent a location on the fused ring structure at which substitution of a functional group may occur. Some examples of such functional groups that may be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, hydroxy groups, phosphorous groups, etc. Functional groups that result in an ionizing capability are often referred to as “chromophores.” Substitution of the ring structure with a chromophore causes a shift in the absorbance wavelength of the compound. Thus, depending on the type of chromophore (e.g., hydroxyl, carboxyl, amino, etc.) and the extent of substitution, a wide variety of anthraquinones may be formed with varying colors and intensities. Other functional groups, such as sulfonic acids, may also be used to render certain types of compounds (e.g., higher molecular weight anthraquinones) water-soluble.

Anthraquinones may be classified for identification by their Color Index (CI) number, which is sometimes called a “standard.” For instance, some suitable anthraquinones that may be used in the present invention, as classified by their “CI” number, include Acid Black 48, Acid Blue 25 (D&C Green No. 5), Acid Blue 40, Acid Blue 41, Acid Blue 45, Acid Blue 80, Acid Blue 129, Acid Green 25, Acid Green 27, Acid Green 41, Acid Violet 43, Mordant Red 11 (Alizarin), Mordant Black 13 (Alizarin Blue Black B), Mordant Red 3 (Alizarin Red S), Mordant Violet 5 (Alizarin Violet 3R), Alizarin Complexone, Natural Red 4 (Carminic Acid), Disperse Blue 1, Disperse Blue 3, Disperse Blue 14, Natural Red 16 (Purpurin), Natural Red 8, Reactive Blue 2 (Procion Blue HB), Reactive Blue 19 (Remazol Brilliant Blue R); and so forth. The structures of Acid Blue 25, Acid Green 41, Acid Blue 45, Mordant Violet 5, Acid Blue 129, Acid Green 25, and Acid Green 27 are set forth below:

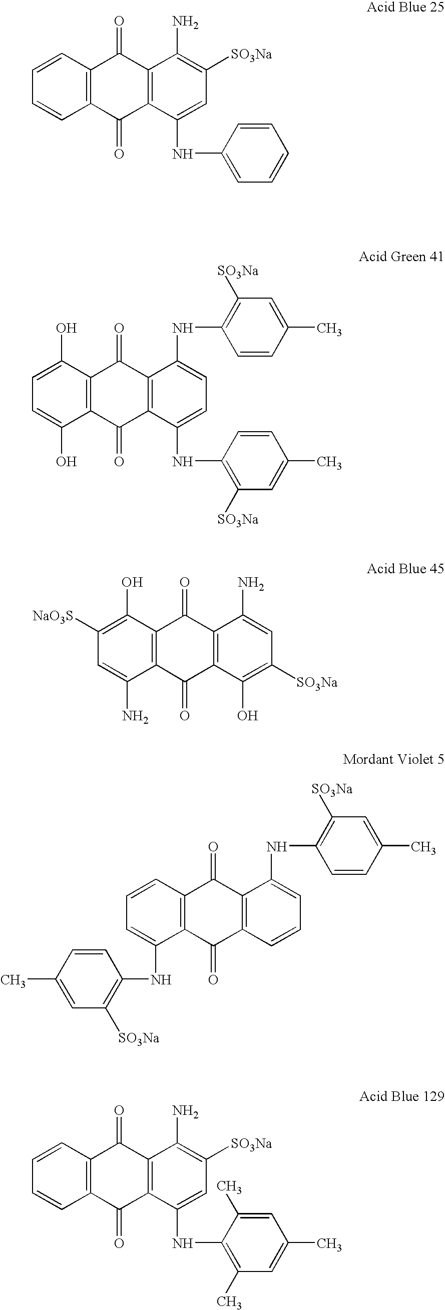

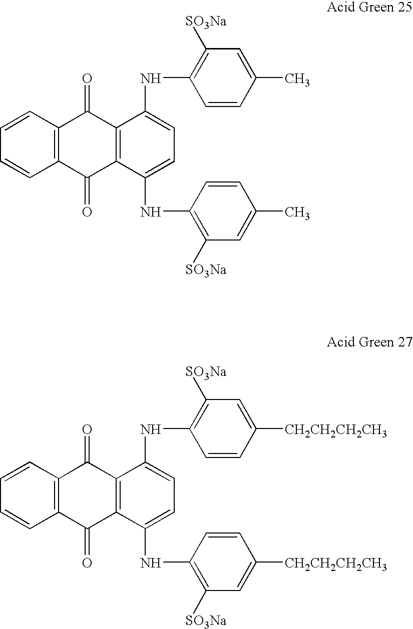

Without intending to be limited by theory, it is believed that the odor caused by many compounds is eliminated by the transfer of electrons to and/or from the malodorous compound. Specifically, oxidation of malodorous compounds via a reduction/oxidation (“redox”) reaction is believed to inhibit the production of the characteristic odor associated therewith. The discovery that certain anthraquinones are able to eliminate odor is believed to be due to their ability to function as an oxidizing agent in a redox reaction. Many common odorous compounds are capable of oxidizing (i.e., donate electrons) via a redox reaction. For instance, odorous compounds may include mercaptans (e.g., ethyl mercaptan), ammonia, amines (e.g., trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.) carboxylic acids (e.g., isovaleric acid, acetic acid, propionic acid, etc.), aldehydes, terpenoids, hexanol, heptanal, pyridine, and so forth. Upon oxidation, the odors associated with such compounds are often eliminated or at least lessened. It is also believed that the reduction of the anthraquinone via the redox reaction is readily reversible, and thus the reduced anthraquinone may be re-oxidized by any known oxidizing agent (e.g., oxygen, air, etc.). The reduction/oxidation reactions are rapid and may take place at room temperature. Thus, although the odor control mechanism may consume the anthraquinones, they may simply be regenerated by exposure to air. Thus, long-term odor control may be achieved without significantly affecting the ability of the anthraquinone to impart the desired color.

The ability of anthraquinones to accept electrons from another substance (i.e., be reduced) may be quantified using a technique known as redox potentiometry. Redox potentiometry is a technique that measures (in volts) the affinity of a substance for electrons—its electronegativity—compared with hydrogen (which is set at 0). Substances more strongly electronegative than (i.e., capable of oxidizing) hydrogen have positive redox potentials. Substances less electronegative than (i.e., capable of reducing) hydrogen have negative redox potentials. The greater the difference between the redox potentials of two substances (ΔE), the greater the vigor with which electrons will flow spontaneously from the less positive to the more positive (more electronegative) substance. As is well known in the art, redox potential may be measured using any of a variety of commercially available meters, such as an Oxidation Reduction Potential (ORP) tester commercially available from Hanna Instruments, Inc. of Woonsocket, R.I. The redox potential of the anthraquinones may, for instance, be less than about −50 millivolts (mV), in some embodiments less than about −150 mV, in some embodiments less than about −300 mV, and in some embodiments, less than about −500 mV. Although not always the case, the redox potential may vary based on the number and location of functional groups, such as sulfonic acid, on the anthraquinone structure. For example, 2-sulfonic acid anthraquinone has a redox potential of −380 mV; 2,6-disulfonic acid anthraquinone has a redox potential of −325 mV; and 2,7-disulfonic acid anthraquinone has a redox potential of −313 mV. The use of other functional groups may also have an affect on the ultimate redox potential of the compound. For example, Acid Blue 25, which also contains amino- and aramid functional groups, has a redox potential of −605 mV.

In addition to their ability to oxidize malodorous compounds, the chemical structure of certain anthraquinones may help improve odor elimination. For example, anthraquinones that have at least one unsubstituted ring may result in better odor inhibition than those that are substituted at each ring with a functional group. Interestingly, anthraquinones that are unsubstituted at the “first” ring (i.e., positions 5 through 8) appear to be particularly effective in reducing odor. Suitable examples of anthraquinones that are unsubstituted at locations at their first ring include, but are not limited to, Acid Blue 25, Acid Blue 129, Acid Green 25, and Acid Green 27, the structures of which are set forth above. Other exemplary odor control anthraquinones are described in U.S. patent application Publication No. 2005/0131363 to MacDonald, et al., which is incorporated herein in its entirety by reference thereto for all purposes.

The odor-reducing anthraquinone may be used alone or in conjunction with other components. For example, nanoparticles, such as described above, may be employed in some embodiments that act as a carrier for the compound. The anthraquinone is believed to form a coordinate bond with an atom of certain nanoparticles (e.g., aluminum) via oxygen atoms present in the anthraquinone structure. As used herein, a “coordinate bond” refers to a shared pair of electrons between two atoms, wherein one atom supplies both electrons to the pair. When utilized, the amount of nanoparticles may generally vary in relation to the anthraquinone. For example, the molar ratio of the nanoparticles to the anthraquinone may range from about 10 to about 10,000, in some embodiments from about 50 to about 5,000, and in some embodiments, from about 100 to about 1,000.

Other than odor control agent(s), the deodorizing ink may also contain other components to facilitate application of the ink to a release liner. For example, the ink may contain a binder for increasing the durability of the ink on the liner, even when present at high levels. Suitable binders may include, for instance, those that become insoluble in water upon crosslinking. Crosslinking may be achieved in a variety of ways, including by reaction of the binder with a polyfunctional crosslinking agent. Examples of such crosslinking agents include, but are not limited to, dimethylol urea melamine-formaldehyde, urea-formaldehyde, polyamide epichlorohydrin, etc. In some embodiments, a polymer latex may be employed as the binder. The polymer suitable for use in the lattices typically has a glass transition temperature of about 30° C. or less so that the flexibility of the resulting liner is not substantially restricted. Moreover, the polymer also typically has a glass transition temperature of about −25° C. or more to minimize the tackiness of the polymer latex. For instance, in some embodiments, the polymer has a glass transition temperature from about −15° C. to about 15° C., and in some embodiments, from about −10° C. to about 0° C. For instance, some suitable polymer lattices that may be utilized in the present invention may be based on polymers such as, but are not limited to, styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, styrene-acrylic copolymers (e.g., Jonrez FV2080, available from MeadWestvaco Corporation, Charleston S.C.), nitrile polymers, and any other suitable anionic polymer latex polymers known in the art. The charge of the polymer lattices described above may be readily varied, as is well known in the art, by utilizing a stabilizing agent having the desired charge during preparation of the polymer latex. For instance, specific techniques for an activated carbon/polymer latex system are described in more detail in U.S. Pat. No. 6,573,212 to McCrae, et al. Activated carbon/polymer latex systems that may be used in the present invention include Nuchar® PMA, DPX-8433-68A, and DPX-8433-68B, all of which are available from MeadWestvaco Corp of Covington, Va.

Although polymer lattices may be effectively used as binders in the present invention, such compounds sometimes result in a reduction in drapability and an increase in residual odor. Thus, water-soluble organic polymers may also be employed as binders to alleviate such concerns. One class of water-soluble organic polymers found to be suitable in the present invention is polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth. Suitable cellulosic ethers may include, for instance, those available from Akzo Nobel of Covington, Va. under the name “BERMOCOLL.” Still other suitable cellulosic ethers are those available from Shin-Etsu Chemical Co., Ltd. of Tokyo, Japan under the name “METOLOSE”, including METOLOSE Type SM (methycellulose), METOLOSE Type SH (hydroxypropylmethyl cellulose), and METOLOSE Type SE (hydroxyethylmethyl cellulose). One particular example of a suitable nonionic cellulosic ether is ethyl hydroxyethyl cellulose having a degree of ethyl substitution (DS) of 0.8 to 1.3 and a molar substitution (MS) of hydroxyethyl of 1.9 to 2.9. The degree of ethyl substitution represents the average number of hydroxyl groups present on each anhydroglucose unit that have been reacted, which may vary between 0 and 3. The molar substitution represents the average number of hydroxethyl groups that have reacted with each anhydroglucose unit. One such cellulosic ether is BERMOCOLL E 230FQ, which is an ethyl hydroxyethyl cellulose commercially available from Akzo Nobel. Other suitable cellulosic ethers are also available from Hercules, Inc. of Wilmington, Del. under the name “CULMINAL.”

The ink may also include various other components as is well known in the art, such as colorants, colorant stabilizers, photoinitiators, binders, solvents, surfactants, humectants, biocides or biostats, electrolytic salts, pH adjusters, etc. For example, various components for use in an ink are described in U.S. Pat. No. 5,681,380 to Nohr, et al. and U.S. Pat. No. 6,542,379 to Nohr, et al., which are incorporated herein in their entirety by reference thereto for all purposes. Examples of suitable humectants include, for instance, ethylene glycol; diethylene glycol; glycerine; polyethylene glycol 200, 400, and 600; propane 1,3 diol; propylene-glycolmonomethyl ethers, such as Dowanol PM (Gallade Chemical Inc., Santa Ana, Calif.); polyhydric alcohols; or combinations thereof. Other additives may also be included to improve ink performance, such as a chelating agent to sequester metal ions that could become involved in chemical reactions over time, a corrosion inhibitor to help protect metal components of the printer or ink delivery system, a biocide or biostat to control unwanted bacterial, fungal, or yeast growth in the ink, and a surfactant to adjust the ink surface tension.

To form the deodorizing ink, its components are first typically dissolved or dispersed in a solvent. For example, one or more of the above-mentioned components may be mixed with a solvent, either sequentially or simultaneously, to form an ink that may be easily applied to a liner. Any solvent capable of dispersing or dissolving the components is suitable, for example water; alcohols such as ethanol or methanol; dimethylformamide; dimethyl sulfoxide; hydrocarbons such as pentane, butane, heptane, hexane, toluene and xylene; ethers such as diethyl ether and tetrahydrofuran; ketones and aldehydes such as acetone and methyl ethyl ketone; acids such as acetic acid and formic acid; and halogenated solvents such as dichloromethane and carbon tetrachloride; as well as mixtures thereof. The concentration of solvent in the ink is generally high enough to allow easy application, handling, etc. If the amount of solvent is too large, however, the amount of odor control agent deposited on the liner might be too low to provide the desired odor reduction. Although the actual concentration of solvent employed will generally depend on the type of odor control agent and the liner on which it is applied, it is nonetheless typically present in an amount from about 40 wt. % to about 99 wt. %, in some embodiments from about 50 wt. % to about 95 wt. %, and in some embodiments, from about 60 wt. % to about 90 wt. % of the ink (prior to drying).

The solids content and/or viscosity of the ink may be varied to achieve the extent of odor reduction desired. For example, the ink may have a solids content of from about 5% to about 90%, in some embodiments from about 10% to about 80%, and in some embodiments, from about 20% to about 70%. By varying the solids content of the ink, the presence of the odor control agent and other components in the deodorizing ink may be controlled. For example, to form a deodorizing ink with a higher level of odor control agent, the ink may be provided with a relatively high solids content so that a greater percentage of the particles are incorporated into the deodorizing ink during the application process. Generally, the viscosity is less than about 2×10⁶ centipoise, in some embodiments less than about 2×10⁵ centipoise, in some embodiments less than about 2×10⁴ centipoise, and in some embodiments, less than about 2×10³ centipoise, such as measured with a Brookfield viscometer, type DV-I or LV-IV, at 60 rpm and 20° C. If desired, thickeners or other viscosity modifiers may be employed in the ink to increase or decrease viscosity.

A variety of techniques may be used for applying the deodorizing ink to the liner. For instance, the ink may be applied using rotogravure or gravure printing, either direct or indirect (offset). Gravure printing encompasses several well-known engraving techniques, such as mechanical engraving, acid-etch engraving, electronic engraving and ceramic laser engraving. Such printing techniques provide excellent control of the composition distribution and transfer rate. Gravure printing may provide, for example, from about 10 to about 1000 deposits per lineal inch of surface, or from about 100 to about 1,000,000 deposits per square inch. Each deposit results from an individual cell on a printing roll, so that the density of the deposits corresponds to the density of the cells. A suitable electronic engraved example for a primary delivery zone is about 200 deposits per lineal inch of surface, or about 40,000 deposits per square inch. By providing such a large number of small deposits, the uniformity of the deposit distribution may be enhanced. Also, because of the large number of small deposits applied to the surface of the liner, the deposits more readily resolidify on the exposed fiber portions. Suitable gravure printing techniques are also described in U.S. Pat. No. 6,231,719 to Garvey, et al., which is incorporated herein in its entirety by reference thereto for all purposes. Moreover, besides gravure printing, it should be understood that other printing techniques, such as flexographic printing, may also be used to apply the ink.

Still another suitable contact printing technique that may be utilized in the present invention is “screen printing.” Screen printing is performed manually or photomechanically. The screens may include a silk or nylon fabric mesh with, for instance, from about 40 to about 120 openings per lineal centimeter. The screen material is attached to a frame and stretched to provide a smooth surface. The stencil is applied to the bottom side of the screen, i.e., the side in contact with the liner upon which the fluidic channels are to be printed. The ink is painted onto the screen, and transferred by rubbing the screen (which is in contact with the liner) with a squeegee.

Ink-jet printing techniques may also be employed in the present invention. Ink-jet printing is a non-contact printing technique that involves forcing the ink through a tiny nozzle (or a series of nozzles) to form droplets that are directed toward the liner. Two techniques are generally utilized, i.e., “DOD” (Drop-On-Demand) or “continuous” ink-jet printing. In continuous systems, ink is emitted in a continuous stream under pressure through at least one orifice or nozzle. The stream is perturbed by a pressurization actuator to break the stream into droplets at a fixed distance from the orifice. DOD systems, on the other hand, use a pressurization actuator at each orifice to break the ink into droplets. The pressurization actuator in each system may be a piezoelectric crystal, an acoustic device, a thermal device, etc. The selection of the type of ink jet system varies on the type of material to be printed from the print head. For example, conductive materials are sometimes required for continuous systems because the droplets are deflected electrostatically. Thus, when the sample channel is formed from a dielectric material, DOD printing techniques may be more desirable.

In addition to the printing techniques mentioned above, any other suitable application technique may be used in the present invention. For example, other suitable printing techniques may include, but not limited to, such as laser printing, thermal ribbon printing, piston printing, spray printing, flexographic printing, etc. Still other suitable application techniques may include bar, roll, knife, curtain, spray, slot-die, dip-coating, drop-coating, extrusion, stencil application, etc. Such techniques are well known to those skilled in the art.

Regardless of the method of application, the deodorizing release liner may be dried at a certain temperature to drive the solvent from the deodorizing ink. For example, the liner may be heated to a temperature of at least about 50° C., in some embodiments at least about 70° C., and in some embodiments, at least about 80° C. By minimizing the amount of solvent in the deodorizing ink, a larger surface area of the odor control agent may be available for contacting malodorous compounds, thereby enhancing odor reduction. It should be understood, however, that relatively small amounts of solvent may still be present. For example, the dried ink may contain a solvent in an amount less than about 10% by weight, in some embodiments less than about 5% by weight, and in some embodiments, less than about 1% by weight.

When dried, the relative percentages and solids add-on level of the resulting odor control agent may vary to achieve the desired level of odor control. The “solids add-on level” is determined by subtracting the weight of the untreated liner from the weight of the treated liner (after drying), dividing this calculated weight by the weight of the untreated liner, and then multiplying by 100%. One particular benefit of the present invention is that high solids add-on levels are achievable without a substantial sacrifice in durability of the ink. In some embodiments, for example, the add-on level of the ink is at least about 2%, in some embodiments from about 4% to about 40%, and in some embodiments, from about 6% to about 35%. The concentration of the odor control agent in the deodorizing ink is generally tailored to facilitate odor control without adversely affecting other properties of the liner, such as flexibility, peelability, etc. For instance, the odor control agent may be present in the ink (prior to drying) in an amount of about 10 wt. % or more, in some embodiments from about 15 wt. % to about 98 wt. %, and in some embodiments, from about 20 wt. % to about 95 wt. %. Other components, such as a binder, may each be present in the ink (after drying) in an amount of from about 10 wt. % to about 80 wt. %, in some embodiments from about 20 wt. % to about 65 wt. %, and in some embodiments, from about 30 wt. % to about 50 wt. %.

The deodorizing ink may be cover an entire surface of the liner, or it may be applied in a pattern. For example, the pattern may cover from about 10% to about 95%, in some embodiments from about 12% to about 90%, and in some embodiments, from about 15% to about 50% of the area of a surface of the liner. The patterned application of the deodorizing ink may provide a variety of benefits, such as presenting a stark and highly visible contrast against a different color (e.g., the color of the background) and thus changing the overall appearance of the liner. For example, the deodorizing ink may have a dark color (e.g., black) and applied against a contrasting light background. Alternatively, a differently colored foreground may contrast with a dark background provided by the deodorizing ink. The relative degree of contrast between the deodorizing ink and the other color may be measured through a gray-level difference value. In a particular embodiment, the contrast may have a gray level value of about 45 on a scale of 0 to about 255, where 0 represents “black” and 255 represents “white.” The analysis method may be made with a Quantimet 600 Image Analysis System (Leica, Inc., Cambridge, UK). This system's software (QWIN Version 1.06A) enables a program to be used in the Quantimet User Interactive Programming System (QUIPS) to make the gray-level determinations. A control or “blank” white-level may be set using undeveloped Polaroid photographic film. An 8-bit gray-level scale may then be used (0-255) and the program allowed the light level to be set by using the photographic film as the standard. A region containing the other color (e.g., background or foreground) may then be measured for its gray-level value, followed by the same measurement of the ink. The routine may be programmed to automatically calculate the gray-level value of the deodorizing ink. The difference in gray-level value between the deodorizing ink and the other color may be about 45 or greater on a scale of 0-255, where 0 represents “black” and 255 represents “white.” The particular type or style of deodorizing ink pattern may include any arrangement of stripes, bands, dots, or other geometric shape. The pattern may include indicia (e.g., trademarks, text, and logos), floral designs, abstract designs, any configuration of artwork, etc.

The patterned application of deodorizing ink may also have various other functional benefits, including optimizing flexibility, peelability, or some other characteristic of the liner. The patterned application of deodorizing ink may provide different odor control properties to multiple locations of the liner. For example, in one embodiment, the liner may be treated with two or more regions of deodorizing ink that may or may not overlap. The regions may be on the same or different surfaces of the liner. In one embodiment, one region of a liner is coated with a first deodorizing ink, while another region is coated with a second deodorizing ink. If desired, one region may be configured to reduce one type of odor, while another region may be configured to reduce another type of odor. Alternatively, one region may possess a higher level of a deodorizing ink than another region or liner to provide different levels of odor reduction.

B. Release Coating

In addition to the deodorizing ink, the release liner of the present invention may also contain a release coating that enhances the ability of the liner to be peeled from an adhesive. The release coating contains a release agent, such as a hydrophobic polymer. Exemplary hydrophobic polymers include, for instance, silicones (e.g., polysiloxanes, epoxy silicones, etc.), perfluoroethers, fluorocarbons, polyurethanes, and so forth. Examples of such release agents are described, for instance, in U.S. Pat. No. 6,530,910 to Pomplun, et al.; U.S. Pat. No. 5,985,396 to Kerins, et al.; and U.S. Pat. No. 5,981,012 to Pomplun, et al., which are incorporated herein in their entirety by reference thereto for all purposes. One particularly suitable release agent is an amorphous polyolefin having a melt viscosity of about 400 to about 10,000 cps at 190° C., such as made by the U.S. Rexene Company under the tradename REXTAC® (e.g., RT2315, RT2535 and RT2330). The release coating may also contain a detackifier, such as a low molecular weight, highly branched polyolefin. A particularly suitable low molecular weight, highly branched polyolefin is VYBAR® 253, which is made by the Petrolite Corporation. Other additives may also be employed in the release coating, such as compatibilizers, processing aids, plasticizers, tackifiers, slip agents, and antimicrobial agents, and so forth.

The release coating may be applied to one or both surfaces of the liner, and may cover all or only a portion of a surface. Although not required, the release coating is typically applied to one surface of the liner and the deodorizing ink is applied to an opposing surface of the liner. In this manner, the release coating may be placed adjacent to an adhesive on the absorbent article and the deodorizing ink may remain visible prior to use. Any suitable technique may be employed to apply the release coating, such as solvent-based coating, hot melt coating, solventless coating, etc. Solvent-based coatings are typically applied to the release liner by processes such as roll coating, knife coating, curtain coating, gravure coating, wound rod coating, and so forth. The solvent (e.g., water) is then removed by drying in an oven, and the coating is optionally cured in the oven. Solventless coatings may include solid compositions, such as silicones or epoxy silicones, which are coated onto the liner and then cured by exposure to ultraviolet light. Optional steps include priming the liner before coating or surface modification of the liner, such as with corona treatment. Hot melt coatings, such as polyethylenes or perfluoroethers, may be heated and then applied through a die or with a heated knife. Hot melt coatings may be applied by co-extruding the release agent with the release liner in blown film or sheet extruder for ease of coating and for process efficiency.

### II. Absorbent Article

The term “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth.

As is well known in the art, the absorbent article may be provided with adhesives (e.g., pressure-sensitive adhesives) that help removably secure the article to the crotch portion of an undergarment and/or wrap up the article for disposal. Suitable pressure-sensitive adhesives, for instance, may include acrylic adhesives, natural rubber adhesives, tackified block copolymer adhesives, polyvinyl acetate adhesives, ethylene vinyl acetate adhesives, silicone adhesives, polyurethane adhesives, thermosettable pressure-sensitive adhesives, such as epoxy acrylate or epoxy polyester pressure-sensitive adhesives, etc. Such pressure-sensitive adhesives are known in the art and are described in the Handbook of Pressure Sensitive Adhesive Technology, Satas (Donatas), 1989, 2^{nd} edition, Van Nostrand Reinhold. The pressure sensitive adhesives may also include additives such as cross-linking agents, fillers, gases, blowing agents, glass or polymeric microspheres, silica, calcium carbonate fibers, surfactants, and so forth. The additives are included in amounts sufficient to affect the desired properties.

The location of the adhesive on the absorbent article is not critical and may vary widely depending on the intended use of the article. For example, certain feminine hygiene products (e.g., sanitary napkins) may have wings or flaps that laterally from a central absorbent core and are intended to be folded around the edges of the wearer's panties in the crotch region. The flaps may be provided with an adhesive (e.g., pressure-sensitive adhesive) for affixing the flaps to the underside of the wearer's panties. Regardless of the particular location of the adhesive, however, the deodorizing release liner of the present invention may be employed to cover the adhesive, thereby protecting it from dirt, drying out, and premature sticking prior to use.

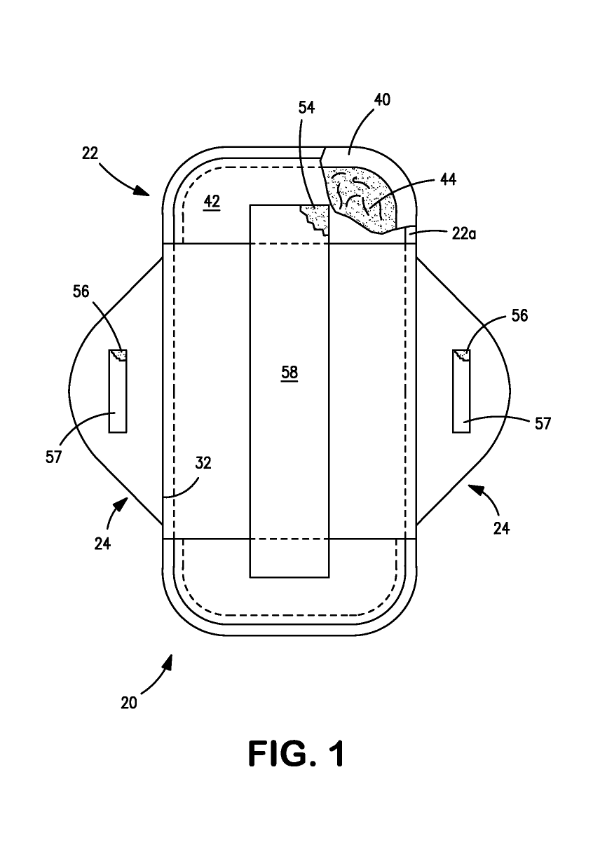

In this regard, various embodiments of an absorbent article that may be formed according to the present invention will now be described in more detail. For purposes of illustration only, an absorbent article 20 is shown in FIG. 1 as a sanitary napkin for feminine hygiene. In the illustrated embodiment, the absorbent article 20 includes a main body portion 22 containing a topsheet 40, an outer cover or backsheet 42, an absorbent core 44 positioned between the backsheet 42 and the topsheet 40, and a pair of flaps 24 extending from each longitudinal side 22a of the main body portion 22. The topsheet 40 defines a bodyfacing surface of the absorbent article 20. The absorbent core 44 is positioned inward from the outer periphery of the absorbent article 20 and includes a body-facing side positioned adjacent the topsheet 40 and a garment-facing surface positioned adjacent the backsheet 42.

The topsheet 40 is generally designed to contact the body of the user and is liquid-permeable. The topsheet 40 may surround the absorbent core 44 so that it completely encases the absorbent article 20. Alternatively, the topsheet 40 and the backsheet 42 may extend beyond the absorbent core 44 and be peripherally joined together, either entirely or partially, using known techniques. Typically, the topsheet 40 and the backsheet 42 are joined by adhesive bonding, ultrasonic bonding, or any other suitable joining method known in the art. The topsheet 40 is sanitary, clean in appearance, and somewhat opaque to hide bodily discharges collected in and absorbed by the absorbent core 44. The topsheet 40 further exhibits good strike-through and rewet characteristics permitting bodily discharges to rapidly penetrate through the topsheet 40 to the absorbent core 44, but not allow the body fluid to flow back through the topsheet 40 to the skin of the wearer. For example, some suitable materials that may be used for the topsheet 40 include nonwoven materials, perforated thermoplastic films, or combinations thereof. A nonwoven fabric made from polyester, polyethylene, polypropylene, bicomponent, nylon, rayon, or like fibers may be utilized. For instance, a white uniform spunbond material is particularly desirable because the color exhibits good masking properties to hide menses that has passed through it. U.S. Pat. No. 4,801,494 to Datta, et al. and U.S. Pat. No. 4,908,026 to Sukiennik, et al. teach various other cover materials that may be used in the present invention.

The topsheet 40 may also contain a plurality of apertures (not shown) formed therethrough to permit body fluid to pass more readily into the absorbent core 44. The apertures may be randomly or uniformly arranged throughout the topsheet 40, or they may be located only in the narrow longitudinal band or strip arranged along the longitudinal axis X-X of the absorbent article 20. The apertures permit rapid penetration of body fluid down into the absorbent core 44. The size, shape, diameter and number of apertures may be varied to suit one's particular needs.

As stated above, the absorbent article also includes a backsheet 42. The backsheet 42 is generally liquid-impermeable and designed to face the inner surface, i.e., the crotch portion of an undergarment (not shown). The backsheet 42 may permit a passage of air or vapor out of the absorbent article 20, while still blocking the passage of liquids. Any liquid-impermeable material may generally be utilized to form the backsheet 42. For example, one suitable material that may be utilized is a microembossed polymeric film, such as polyethylene or polypropylene. In particular embodiments, a polyethylene film is utilized that has a thickness in the range of about 0.2 mils to about 5.0 mils, and particularly between about 0.5 to about 3.0 mils.

The absorbent article 20 also contains an absorbent core 44 positioned between the topsheet 40 and the backsheet 42. The absorbent core 44 may be formed from a single absorbent member or a composite containing separate and distinct absorbent members. It should be understood, however, that any number of absorbent members may be utilized in the present invention. For example, in one embodiment, the absorbent core 44 may contain an intake member (not shown) positioned between the topsheet 40 and a transfer delay member (not shown). The intake member may be made of a material that is capable of rapidly transferring, in the z-direction, body fluid that is delivered to the topsheet 40. The intake member may generally have any shape and/or size desired. In one embodiment, the intake member has a rectangular shape, with a length equal to or less than the overall length of the absorbent article 20, and a width less than the width of the absorbent article 20. For example, a length of between about 150 mm to about 300 mm and a width of between about 10 mm to about 60 mm may be utilized.

Any of a variety of different materials are capable of being used for the intake member to accomplish the above-mentioned functions. The material may be synthetic, cellulosic, or a combination of synthetic and cellulosic materials. For example, airlaid cellulosic tissues may be suitable for use in the intake member. The airlaid cellulosic tissue may have a basis weight ranging from about 10 grams per square meter (gsm) to about 300 gsm, and in some embodiments, between about 100 gsm to about 250 gsm. In one embodiment, the airlaid cellulosic tissue has a basis weight of about 200 gsm. The airlaid tissue may be formed from hardwood and/or softwood fibers. The airlaid tissue has a fine pore structure and provides an excellent wicking capacity, especially for menses.

If desired, a transfer delay member (not shown) may be positioned vertically below the intake member. The transfer delay member may contain a material that is less hydrophilic than the other absorbent members, and may generally be characterized as being substantially hydrophobic. For example, the transfer delay member may be a nonwoven fibrous web composed of a relatively hydrophobic material, such as polypropylene, polyethylene, polyester or the like, and also may be composed of a blend of such materials. One example of a material suitable for the transfer delay member is a spunbond web composed of polypropylene, multi-lobal fibers. Further examples of suitable transfer delay member materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay member are described in U.S. Pat. No. 4,798,603 to Meyer, et al. and U.S. Pat. No. 5,248,309 to Serbiak, et al., which are incorporated herein in their entirety by reference thereto for all purposes. To adjust the performance of the invention, the transfer delay member may also be treated with a selected amount of surfactant to increase its initial wettability.

The transfer delay member may generally have any size, such as a length of about 150 mm to about 300 mm. Typically, the length of the transfer delay member is approximately equal to the length of the absorbent article 20. The transfer delay member may also be equal in width to the intake member, but is typically wider. For example, the width of the transfer delay member may be from between about 50 mm to about 75 mm, and particularly about 48 mm. The transfer delay member typically has a basis weight less than that of the other absorbent members. For example, the basis weight of the transfer delay member is typically less than about 150 grams per square meter (gsm), and in some embodiments, between about 10 gsm to about 100 gsm. In one particular embodiment, the transfer delay member is formed from a spunbonded web having a basis weight of about 30 gsm.

Besides the above-mentioned members, the absorbent core 44 may also include a composite absorbent member (not shown), such as a coform material. In this instance, fluids may be wicked from the transfer delay member into the composite absorbent member. The composite absorbent member may be formed separately from the intake member and/or transfer delay member, or may be formed simultaneously therewith. In one embodiment, for example, the composite absorbent member may be formed on the transfer delay member or intake member, which acts a carrier during the coform process described above.

Regardless of its particular construction, the absorbent article 20 typically contains an adhesive for securing to an undergarment. An adhesive may be provided at any location of the absorbent article 20, such as on the lower surface of the backsheet 42. In this particular embodiment, the backsheet 42 carries a longitudinally central strip of garment adhesive 54 covered before use by a peelable release liner 58, which may be formed in accordance with the present invention. Each of the flaps 24 may also contain an adhesive 56 positioned adjacent to the distal edge 34 of the flap 24. A peelable release liner 57, which may also be formed in accordance with the present invention, may cover the adhesive 56 before use. Thus, when a user of the sanitary absorbent article 20 wishes to expose the adhesives 54 and 56 and secure the absorbent article 20 to the underside of an undergarment, the user simply peels away the liners 57 and 58. Once removed, the release liners 57 and/or 58 may be disposed, either alone or in conjunction with a used absorbent article. Many absorbent articles (e.g., feminine hygiene products), for example, are disposed by placing them in a small pouch in which the product is packaged for sale. If desired, the deodorizing release liner of the present invention may be disposed in the pouch to help reduce odors associated with the disposed absorbent articles. Various suitable pouch configurations are disclosed in U.S. Pat. No. 6,716,203 to Sorebo, et al. and U.S. Pat. No. 6,380,445 to Moder, et al., as well as U.S. patent application Publication No. 2003/0116462 to Sorebo, et al., all of which are incorporated herein in their entirety by reference thereto for all purposes.

Although various embodiments of an absorbent article have been described above that may incorporate the benefits of the present invention, it should be understood that other configurations are also included within the scope of the present invention. For instance, other absorbent article configurations are described in U.S. Pat. No. 5,649,916 to DiPalma, et al.; U.S. Pat. No. 6,110,158 to Kielpikowski; U.S. Pat. No. 6,663,611 to Blaney, et al.; U.S. Pat. No. 4,886,512 to Damico et al.; U.S. Pat. No. 5,558,659 to Sherrod et al.; U.S. Pat. No. 6,888,044 to Fell et al.; and U.S. Pat. No. 6,511,465 to Freiburger et al., as well as U.S. patent application Publication No. 2004/0060112 A1 to Fell et al., all of which are incorporated herein in their entirety by reference thereto for all purposes.

The effectiveness of the deodorizing release liner of the present invention may be measured in a variety of ways. For example, the percent of a malodorous compound adsorbed by the deodorizing ink may be determined in accordance with the headspace gas chromatography test set forth herein. In some embodiments, for instance, the release liner is capable of adsorbing at least about 25%, in some embodiments at least about 45%, and in some embodiments, at least about 65% of a particular malodorous compound, such as mercaptans (e.g., ethyl mercaptan), ammonia, amines (e.g., trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.) carboxylic acids (e.g., isovaleric acid, acetic acid, propionic acid, etc.), aldehydes, terpenoids, hexanol, heptanal, pyridine, and so forth. The effectiveness of the ink in removing odors may also be measured in terms of “Relative Adsorption Efficiency”, which is determined using headspace gas chromatography and measured in terms of milligrams of odor adsorbed per gram of the ink. It should be recognized that the chemistry of any one type of odor control ink may not be suitable to reduce all types of malodorous compounds, and that low adsorption of one or more malodorous compounds may be compensated by good adsorption of other malodorous compounds.

The present invention may be better understood with reference to the following examples.

### Test Methods

Qualitative and quantitative odor reduction was tested in the Examples. Quantitative odor reduction was determined using a test known as “Headspace Gas Chromatography.” Headspace gas chromatography testing was conducted on an Agilent Technologies 5890, Series II gas chromatograph with an Agilent Technology 7694 headspace sampler (Agilent Technologies, Waldbronn, Germany). Helium was used as the carrier gas (injection port pressure: 87.5 kPa; headspace vial pressure: 108.9 kPa; supply line pressure is at 413.4 kPa). A DB-624 column was used for the malodorous compound that had a length of 30 meters and an internal diameter of 0.25 millimeters. Such a column is available from J&W Scientific, Inc. of Folsom, Calif. The operating parameters used for the headspace gas chromatography are shown below in Table 1:

TABLE 1
Operating Parameters for the Headspace Gas Chromatography
Headspace Parameters

| | | |
|---|---|---|
| Zone Temps, ° C. | Oven | 37 |
| | Loop | 85 |
| | TR. Line | 90 |
| Event Time, minutes | GC Cycle time | 10.0 |
| | Vial eq. Time | 10.0 |
| | Pressuriz. Time | 0.20 |
| | Loop fill time | 0.20 |
| | Loop eq. Time | 0.15 |
| | Inject time | 0.30 |
| Vial Parameters | First vial | 1 |
| | Last vial | 1 |
| | Shake | [off] |

The test procedure involved placing a sample in a headspace vial. Using a syringe, an aliquot of the relevant malodorous compound (ethyl mercaptan or triethylamine) was also placed in the vial. Each sample was tested in triplicate. The vial was then sealed with a cap and a septum and placed in the headspace gas chromatography oven at 37° C. After two (2) hours, a hollow needle was inserted through the septum and into the vial. A 1-cubic centimeter sample of the headspace (air inside the vial) was then injected into the gas chromatograph. Initially, a control vial with only the aliquot of malodorous compound was tested to define 0% malodorous compound adsorption. To calculate the amount of headspace malodorous compound removed by the sample, the peak area for the malodorous compound from the vial with the sample was compared to the peak area from the malodorous compound control vial.

### EXAMPLE 1

A corona-treated polyethylene film (Pliant Corp.) was coated with a thin layer of activated carbon ink on one side using a Mayer rod (#5). Specifically, 10 milliliters of an carbon ink (Nuchar® PMA) was placed along the top of a sample of film (20 cm×30 cm) as a line of liquid. The liquid was drawn down to give a thin film of the ink which was then allowed to air dry overnight at ambient temperature. The weight of the ink was determined to be a 7 grams per square meter. This film was then cut into samples having the size of a release liner, i.e., 6 centimeters by 15 centimeters. To test the release liners, incontinence pads (POISE® Extra Plus) pads were provided that contained 30 milliliters of fresh pooled female urine on the body side of the pad. All of the urine was allowed to absorb into the pad core. The release liner sample was placed on top of the body side of the pad. The pad was then carefully rolled up with the release liner. Control pads, which also contained urine, were rolled up in a similar manner with release liners that had not been coated with deodorizing ink. Each pad was stored in separate mason jars (1 quart size) with a lid and incubated at 37° C. for 24 hours before odor assessment. The odor intensity of each jar was ranked (1 for lowest odor and 10 for greatest odor) by a sensory panel consisting of at least 4 panelists. The jars were wrapped in aluminum foil to ensure the pad could not been seen. The odor of each jar was ranked and the scores were added to give the final ranking. The results are shown below in Table 2.

TABLE 2
Urine Odor Ranking of Carbon Ink

| | Odor Ranking |
|---|---|
| Samples | (4 panelists, summed scores) |
| Control liner | 40 |
| Carbon ink liner | 4 |

The results show the significant odor reduction of the carbon ink-coated release liner compared to the untreated control liner.

### EXAMPLE 2

Modified silica particles were prepared for treatment of a film. The silica particles were Snowtex-OXS, which are colloidal silica nanoparticles commercially available from Nissan Chemical America of Houston, Tex. The particles have an average particle size of between 4 to 6 nanometers. The silica particles were modified with a transition metal as follows. An aqueous solution of iron (III) chloride hexahydrate (FeCl₃.6H₂O) was added to an aqueous solution of Snowtex-OXS to form a suspension having a copper:silica molar ratio of 25:1. The particles were then dispersed into a water solution of 35% wt/wt Bermocoll E 230FQ (Akzo Nobel). This coating was applied to a corona-treated polyethylene film (20 cm×30 cm) by placing approximately 10 milliliters of the solution in a line along the top of the film and using a Meyer rod (#5). The liquid was drawn down over the film to leave a very thin coating of liquid on the film. The coating was allowed to dry overnight at ambient temperature. The film was then formed into release liner samples and tested as described in Example 1, except that incubation was conducted for only 12 hours. The results are set forth below in Table 3.

TABLE 3
Urine Odor Ranking of Copper/Silica Ink

| | Odor Ranking |
|---|---|
| Sample | (4 panelists, summed scores) |
| Control | 40 |
| Copper/Silica liner | 4 |

The results show the significant odor reduction of the copper/silica ink-coated release liner compared to the untreated control liner.

### EXAMPLE 3

A 50-milliliter water solution of 35% wt/wt Bermocoll E 230FQ (Akzo Nobel) containing 5 milliliters of isopropanol was prepared. Upon formation, 0.5 grams of D&C Green 5 dye was added to the solution, following by stirring for 10 minutes to ensure homogeneity. This ink was then applied to a corona-treated polyethylene film via a Meyer rod. Approximately 10 milliliters was placed along the top of the film sample (20 cm×30 cm) as a line of liquid. The liquid was then drawn down across the film using a Meyer rod (#5) and the ink allowed to dry overnight at ambient temperature. The film was then formed into release liner samples and tested as described in Example 1, except that incubation was conducted for only 12 hours. The results are set forth below in Table 4.

TABLE 4
Urine Odor Ranking of Anthraquinone Ink

| | Odor Ranking |
|---|---|
| Sample | (4 panelists, summed scores) |
| Control | 40 |
| D&C Green liner | 4 |

The results show the significant odor reduction of the anthraquinone ink-coated release liner compared to the untreated control liner.

### EXAMPLE 4

The ink of Example 3 was applied as stripes to a polypropylene spunbond web (basis weight of 2 ounces per square yard, size of 20 cm×30 cm). The stripes were formed with a pipette containing the D&C Green 5 solution. The solution was slowly released onto the spunbond web by applying gentle pressure to the pipette bulb. The pipette was run across the top surface of the spunbond while the solution was deposited. A space was allowed before repeating the process to give a series of parallel lines (stripes) across the fabric. The fabric had approximately 50% coverage of the ink. The ink was allowed to dry overnight and then cut up into strips (6 cm×15 cm) where the stripes ran perpendicular to the rectangular length of the release liner. The strips were then tested as described in Example 1, except that the incubation time was only 12 hours. The results are set forth below in Table 5.

TABLE 5
Urine Odor Ranking of Anthraquinone Ink

| | Odor Ranking |
|---|---|
| Sample | (4 panelists, summed scores) |
| Control | 40 |
| Dye striped spunbond | 4 |

### EXAMPLE 5

Modified silica particles were prepared for treatment of a film, which can be used to form a release liner. The silica particles were Snowtex-OXS, which are colloidal silica nanoparticles commercially available from Nissan Chemical America of Houston, Tex. The particles have an average particle size of between 4 to 6 nanometers. The silica particles were modified with a transition metal as follows.

A solution of iron (III) chloride hexahydrate (FeCl₃6H₂O) (78.1 grams, 0.289 moles) in water (500 milliliters) was added to 2.4 liters of an aqueous solution of Snowtex-OXS (10 wt. % solids, 2.89×10⁻³ moles SiO₂ particles). The suspension was stirred until the iron salt dissolved in the solution. Water (2 liters) was then added to the mixture. While vigorously stirring the suspension, a solution of sodium bicarbonate (NaHCO₃) (26.8 grams NaHCO₃ in 3.6 liters of water, 0.32 moles NaHCHO₃) was added. The resulting FeOXS supsension was stirred at room temperature for 1 hour. A corona-treated polyethylene film (Pliant Corp.) was then laid flat onto an Accu-Lab™ Drawdown Machine (UV Process Supply, Inc.; Chicago, Ill.). The FeOXS suspension (2 milliliters) was transferred to one end of the film and evenly spread over the entire film surface using a pull down bar (with grooves) and moving it in a single direction. The treated film was allowed to dry in air.

The ink-coated film was then assessed for its ability to adsorb ethyl mercaptan using the above-described headspace gas chromatography (GC) test. Specifically, three (3) strips of the FeOXS treated PE film (0.0738 grams, 0.0750 grams, and 0.0786 grams, respectively) were each transferred to different headspace GC sample vials. Ethyl mercaptan (1 mL, 839 mg) was injected into each sample vial; and the vial was sealed immediately. The sample vials were transferred to the headspace GC instrument for data collection. Three (3) untreated polyethylene film samples (0.0754 g, 0.0713 g, and 0.0742 g) were also assessed for comparison. The results are set fort below in Table 6 in terms of the average milligrams of ethyl mercaptan removed per gram of the sample.

TABLE 6
Removal of Ethyl Mercaptan

| | Avg. Milligrams of Ethyl Mercaptan |
|---|---|
| Sample | Removed per Gram of Sample |
| Treated film | 11.00 |
| Untreated film | 0.48 |

The ink-coated film was also assessed for its ability to adsorb triethylamine using the above-described headspace gas chromatography (GC) test. Specifically, three (3) strips of the FeOXS treated PE film (0.0703 grams, 0.0805 grams, and 0.0798 grams, respectively) were each transferred to different headspace GC sample vials. Triethylamine (1 mL, 726 mg) was injected into each sample vial; and the vial was sealed immediately. The sample vials were transferred to the headspace GC instrument for data collection. Three (3) untreated polyethylene film samples (0.0846 g, 0.0863 g, and 0.0910 g) were also assessed for comparison. The results are set fort below in Table 7 in terms of the milligrams of triethylamine removed per gram of the sample.

TABLE 7
Removal of Triethylamine

| | Avg. Milligrams of Triethylamine |
|---|---|
| Sample | Removed per Gram of Sample |
| Treated film | 8.70 |
| Untreated film | 4.58 |

### EXAMPLE 6

Initially, the following five (5) solutions were formed:
1. An aqueous solution of 2.5% wt/wt D&C Green No. 25 (Sigma-Aldrich Chemical Co., St. Louis, Mo.).
2. An aqueous solution of 2.5% wt/wt D&C Green No. 25 (Sigma-Aldrich Chemical Co., St. Louis, Mo.) and 5.0% wt/wt Snowtex AK nanoparticles (Nissan Chemical America of Houston, Tex.).
3. An aqueous solution of 2% wt/wt BERMOCOLL E 230FQ (Akzo Nobel).
4. An aqueous solution of 17.8% wt/wt calcium carbonate.
5. An aqueous solution of 60% wt/wt of a white flexographic ink (Akzo Nobel).

From these solutions, odor control inks were formed as follows:

| | Solution | Solution | Solution | Solution | Solution |
|---|---|---|---|---|---|
| Ink | No. 1 | No. 2 | No. 3 | No. 4 | No. 5 |
| Sample | (mL) | (mL) | (mL) | (mL) | (mL) |
| A | — | 1.0 | 1.0 | — | 0.5 |
| B | 1.0 | — | 1.0 | — | 0.5 |
| C | — | 1.0 | 1.0 | 1.0 | — |
| D | 1.0 | — | 1.0 | 1.0 | — |
| E | — | 1.0 | 1.0 | — | — |
| F | 1.0 | — | 1.0 | — | — |

Once formed, an aliquot (1 mL) of each ink sample was pipetted onto a corona-treated polyethylene film (Pliant Corp.) in front of an unthreaded metal draw down bar. The bar was then pulled down by hand to draw down the fluid as smoothly as possible. The treated films were allowed to dry in air. Strips were cut from each test sheet and placed in small glass jars with a slice of garlic for overnight incubation at room temperature. The next day, the jars were assessed. It was determined that Sample B (containing D&C Green No. 5, E230 binder, and the flexographic ink) achieved the best odor reduction. The nanoparticles did not appear to provide a significant improvement in odor reduction. A drop of water was also placed onto each of the samples and wiped, which resulted in the removal of the ink for Samples C-F. The films were then placed in an oven to cure for 15 minutes at 80° C. After this time, a drop of water was also placed onto each of the samples and wiped, which again resulted in the removal of the ink for Samples C-F.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An absorbent article comprising a body portion that includes a liquid permeable topsheet, a generally liquid impermeable backsheet, and an absorbent core positioned between the backsheet and the topsheet, the absorbent article further comprising a release liner that defines a first surface and an opposing second surface, the first surface being disposed adjacent to an adhesive located on the absorbent article, wherein the release liner is coated with an ink that contains an odor control agent.

2. The absorbent article of claim 1, wherein the release liner is a paper, film, nonwoven web, or a combination thereof.

3. The absorbent article of claim 1, wherein the odor control agent includes activated carbon particles.

4. The absorbent article of claim 1, wherein the odor control agent includes inorganic oxide nanoparticles.

5. The absorbent article of claim 4, wherein the inorganic oxide nanoparticles include silica, alumina, or a combination thereof.

6. The absorbent article of claim 4, wherein the inorganic oxide nanoparticles are modified with a transition metal.

7. The absorbent article of claim 1, wherein the odor control agent includes an odor-reducing anthraquinone compound having the following general formula: 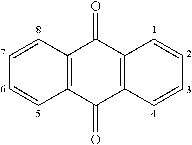
wherein the numbers 1 through 8 refer to optional substitution positions for functional groups.

8. The absorbent article of claim 7, wherein the odor-reducing anthraquinone is selected from the group consisting of Acid Blue 25, Acid Blue 40, Acid Blue 45, Acid Blue 80, Acid Blue 129, Acid Green 25, Acid Green 27, Acid Green 41, D&C Green No. 5, Mordant Violet 5, Mordant Black 13, Reactive Blue 19, and Reactive Blue 2.

9. The absorbent article of claim 1, wherein the odor control agent constitutes about 10 wt. % or more of the ink.

10. The absorbent article of claim 1, wherein the odor control agent constitutes from about 25 wt. % to about 95 wt. % of the ink.

11. The absorbent article of claim 1, wherein the ink further comprises a binder.

12. The absorbent article of claim 11, wherein the binder constitutes from about 10 wt. % to about 80 wt. % of the ink.

13. The absorbent article of claim 1, wherein the ink is coated onto the release liner in a pattern that covers from about 10% to about 95% of the area of a surface of the liner.

14. The absorbent article of claim 1, wherein the ink covers substantially an entire inner surface of the releaser liner.

15. The absorbent article of claim 1, wherein the second surface of the release liner is coated with the ink.

16. The absorbent article of claim 15, wherein a release agent is coated onto the first surface of the release liner.

17. The absorbent article of claim 16, wherein the release agent includes a hydrophobic polymer.

18. The absorbent article of claim 1, wherein the adhesive is a pressure-sensitive adhesive.

19. The absorbent article of claim 1, wherein the adhesive is located on a surface of the backsheet.

20. The absorbent article of claim 1, further comprising at least one flap extending from the body portion, wherein the adhesive is located on a surface of the flap.

21. The absorbent article of claim 1, wherein the absorbent article is a sanitary napkin.

22. A deodorizing release liner that defines a first surface and an opposing second surface, wherein an ink that contains an odor control agent is provided on the first surface of the liner and a coating that comprises a release agent is provided on the second surface of the liner.

23. The release liner of claim 22, wherein the release liner is a paper, film, nonwoven web, or a combination thereof.

24. The release liner of claim 22, wherein the odor control agent includes activated carbon particles.

25. The release liner of claim 22, wherein the odor control agent includes inorganic oxide nanoparticles.

26. The release liner of claim 25, wherein the inorganic oxide nanoparticles include silica, alumina, or a combination thereof.

27. The release liner of claim 25, wherein the inorganic oxide nanoparticles are modified with a transition metal.

28. The release liner of claim 22, wherein the odor control agent includes an odor-reducing anthraquinone compound having the following general formula: 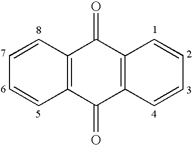
wherein the numbers 1 through 8 refer to optional substitution positions for functional groups.

29. The release liner of claim 28, wherein the odor-reducing anthraquinone is selected from the group consisting of Acid Blue 25, Acid Blue 40, Acid Blue 45, Acid Blue 80, Acid Blue 129, Acid Green 25, Acid Green 27, Acid Green 41, D&C Green No. 5, Mordant Violet 5, Mordant Black 13, Reactive Blue 19, and Reactive Blue 2.

30. The release liner of claim 22, wherein the odor control agent constitutes about 10 wt. % or more of the ink.

31. The release liner of claim 22, wherein the odor control agent constitutes from about 25 wt. % to about 95 wt. % of the ink.

32. The release liner of claim 22, wherein the ink further comprises a binder.

33. The release liner of claim 32, wherein the binder constitutes from about 10 wt. % to about 80 wt. % of the ink.

34. The release liner of claim 22, wherein the release agent includes a hydrophobic polymer.

35. A method for reducing the odor associated with a personal care absorbent article that contains a bodily fluid, the method comprising disposing the article and a release liner into a container, wherein the release liner is coated with an ink that contains an odor control agent, wherein the odor control agent is configured to adsorb a malodorous compound associated with the bodily fluid.

36. The method of claim 35, wherein the malodorous compound is a mercaptan, ammonia, amine, sulfide, ketone, carboxylic acid, aldehyde, terpenoid, hexanol, heptanal, pyridine, or a combination thereof.
